(19) United States
(12) Patent Application Publication
(10) Pub. No.: US 2013/0158388 A1
(43) Pub. Date: Jun. 20, 2013

(54) NEEDLE GUIDANCE FOR MOLECULAR IMAGING

(71) Applicant: Ira Micah Blevis, Tirat Carmel (IL)

(72) Inventor: Ira Micah Blevis, Tirat Carmel (IL)

(73) Assignee: General Electric Company, Schenectady, NY (US)

(21) Appl. No.: 13/330,196

(22) Filed: Dec. 19, 2011

(51) Int. Cl. A61B 6/00; A61B 10/02

(52) U.S. Cl. 600/424

(57) ABSTRACT

A system and method for biopsy or other needle guidance procedures for molecular imaging. A molecular imaging system having one or more radiation detectors for scanning tissue within a region of interest is provided, as well as a hollow needle for performing a medical procedure on the tissue, and a radioisotope in substantially liquid form. The radioisotope is introduced into the lumen of the needle, and the radiation emitted from the radioisotope contained in the needle lumen is detected using the molecular imaging system so that the needle can be guided into the region of interest.

### BACKGROUND OF THE INVENTION

The present invention relates generally to nuclear medicine imaging, and more particularly to medical procedures for nuclear medicine imaging, such as biopsy needle guidance for molecular breast imaging.

In diagnostic medical imaging, there are generally two types of imaging. One is anatomical imaging, which shows the internal physical structure of the anatomy. This type of imaging can be accomplished using x-ray (XR), computed tomography (CT), ultrasound (U/S), magnetic resonance imaging (MRI), and the like. The other type is functional imaging, which shows how various substances flow through the vasculature and/or how they are used or metabolized by the body. This type of imaging can be accomplished using nuclear medicine (NM), single photon emission computer tomography (SPECT), positron emission tomography (PET), functional magnetic resonance imaging (fMRI), and the like.

The NM, SPECT and PET approaches to functional imaging are often grouped together in a category known as molecular imaging. In molecular imaging, a radiopharmaceutical (RP) or radiotracer is injected into a patient (or is ingested or inhaled by the patient) and radioactive particles are emitted by the RP which flows through the patient's bloodstream and becomes distributed throughout the body as a result of metabolism. For example, in PET imaging, the patient is injected with fluorodeoxyglucose (FDG), which is a type of glucose analog that is tagged with radioactive fluorine (¹⁸F). The FDG is taken up throughout the body due to the glucose analog being metabolized. Meanwhile, the ¹⁸F portion of the FDG emits positrons, which upon interaction with surrounding tissue causes 511 keV gamma photons to be emitted, which can then be detected with a PET detector. The PET detector then calculates the locations where the gamma emissions originated from within the body, and displays an image showing how the FDG is distributed throughout the body. Similarly, in NM imaging, a patient undergoing a cardiac examination may be injected with technetium-99m (^{99m}Tc) Sestamibi, which is used for myocardial perfusion imaging. The ^{99m}Tc portion emits 140 keV gamma photons, which can be detected by a gamma camera to form an image showing how well the blood flows through the heart. In each type of molecular imaging, areas showing unusually high uptake of RP may indicate cancerous tissue, since cancer cells generally have a higher rate of metabolism than normal healthy cells.

When molecular imaging procedures are performed and an area of high RP uptake is detected, the clinician may desire to perform a biopsy of the suspicious area. A biopsy is an invasive procedure which extracts a small amount of tissue from the suspicious area so the tissue can be evaluated in a laboratory. Depending on various factors, the biopsy can range from a more invasive procedure which involves surgery, to a minimally invasive procedure like needle biopsy. In needle biopsy, a hollow needle is inserted through the skin and into the suspicious area. There are two types of needle biopsy procedures: fine needle aspiration (FNA) and core needle biopsy. FNA involves a very fine needle which extracts fluid and/or a small amount of cells from the suspicious tissue, while core needle biopsy utilizes a larger needle with a special compartment for capturing a larger sample of tissue.

One approach to core needle biopsies is illustrated in FIG. 1 in four steps. In step (a), a needle 10 having an inner body 12 with a slot or compartment 14 formed therein which is covered by a slideable outer sheath 16 is prepared for insertion into a lesion or suspicious area 20. In step (b), the needle's inner body 12 is inserted into the lesion 20, and a small amount of the lesion tissue 20 fills some or all of the compartment 14. Then, in step (c), the inner body 12 is held in place within the lesion 20 while the other sheath 16 is slid over the inner body 12 so as to close off the compartment 14. The leading edge 18 of the sheath 16 acts to sever a sample 22 of the lesion 20 as the sheath 16 slides to cover the compartment 14. Finally, with the sample 22 captured within the now-covered compartment 14, in step (d) the entire needle 10 is removed from the lesion 20. The sample 22 can now be extracted from the needle 10 for testing by extending the inner body 12, thus exposing the compartment 14 and its sample 22. Alternatively, in step (b), the sheath 16 may be closed (i.e., the compartment 14 is covered by the sheath 16) during insertion. Once inserted, the compartment 14 is then opened by withdrawing the sheath 16 to allow tissue 20 to enter the compartment 14 before proceeding on to step (c). As another alternative, in vacuum-assisted core biopsy, the cut tissue sample 22 is sucked out through a lumen of the needle (not shown), then the sheath 16 is reopened; the needle may be turned or moved slightly to access another volume of tissue, and then another sample 22 is cut by the closing sheath 16. This process may be repeated to obtain a number of samples 22.

One challenge with performing biopsies is that it is difficult to know exactly where within the tissue a lesion or suspicious tissue is situated, and where the needle is in relation to the lesion. This difficulty is often addressed by using a diagnostic imaging procedure (such as x-ray) while the biopsy is being conducted. Often it is desirable to perform the biopsy immediately after a lesion or suspicious area is detected, while the patient is still undergoing the diagnostic imaging procedure which identified the lesion. For example, if a woman is undergoing a diagnostic breast exam using conventional x-ray-based mammography and a potential lesion is detected, the woman may be kept in the mammography machine with her breast compressed between the two paddles (one being an x-ray emitter and the other being an x-ray detector), and a biopsy can immediately be performed using the x-ray image of the breast to guide the needle. (Or, the patient can be brought to another x-ray mammography machine that better facilitates the use of needle biopsy devices.) Since the metal needle has very high attenuation, it shows up easily and clearly on an x-ray image, thus facilitating needle guidance.

However, when molecular breast imaging is performed and a potential lesion is detected, needle guidance using the molecular imaging system is much more difficult than when using x-ray-based imaging. This is because the detectors used in molecular imaging are designed to detect radiation emitters (such as the RPs in the body), and therefore a typical biopsy needle would not be detected by these detectors. More specifically, such detectors are usually designed to optimally detect radiation at certain energies, such as 140 keV for gamma cameras used in NM or 511 keV for detector modules used in PET. Therefore, a normal metal needle would not be detected by a molecular imaging system.

Various prior art approaches have been devised for visualizing the needle used for biopsies during molecular imaging. One approach is to use needles that have at least some portion thereof made of a radioactive material that can be detected by molecular imaging detectors. For example, U.S. Pat. No. 4,781,198 to Kanabrocki (incorporated herein by reference) teaches a stainless steel needle having a layer of radioactive material (such as cobalt 57) electrolessly plated only on the tip of the needle, with a layer of non-radioactive material covering the radioactive material. Likewise, U.S. Pat. No. 5,938,604 to Wagner et al. (incorporated herein by reference), which is assigned to Capintec, Inc. of Ramsey, New Jersey, teaches coating the tip of the needle with a radioactive material, then covering this coating with an intermediate layer of non-radioactive material, and then covering the entire needle with an outer layer of non-radioactive material. A related approach is taught in U.S. Pat. No. 5,647,374 to Cutrer (incorporated herein by reference), in which a wire having a radioactive tip is sealed within a thin elongate tube, with the radioactive tip positioned at a distal end of the tube. The other (proximate) end of the tube is captured by a hub, and together the wire, tube and hub form a stylus which can be inserted into a biopsy needle such that the radioactive wire tip is positioned near the tip of the needle. However, the processes for creating, using, and disposing of such needles or radioactive wires as taught in the above patents are complicated, expensive and use environmentally undesirable processes such as electroplating and chemical deposition.

Another set of approaches, taught in U.S. Pat. No. 5,961,457 to Raylman et al. (incorporated herein by reference), also addresses biopsy needle guidance for molecular imaging. One approach is to administer an RP into a patient, scan the patient to produce sinogram space coordinates of the suspected lesion, convert the sinogram space coordinates into cylindrical coordinates, convert the cylindrical coordinates into Cartesian coordinates, and then use the Cartesian coordinates to guide the biopsy needle to the lesion. The other approach is to obtain radiation emission data of the suspected lesion while generating scanner space coordinates, convert the scanner space coordinates into Cartesian coordinates, and then use the Cartesian coordinates to guide the biopsy needle to the lesion. However, these approaches are complicated and apply to a class of systems capable of producing sinograms, and focus only on how to calculate where the needle should go, without providing a way of actually tracking the position of the needle itself.

Another approach, developed by Naviscan, Inc. of San Diego, Calif. and marketed as Stereo Navigator®, is to compress the breast between two compression paddles, align a needle guide holder (having multiple guide holes therein) with the paddles, and place a needle (comprising an outer sheath and an inner stylet) through one of the guide holes and into the potential lesion within the compressed breast by following software instructions. Then the stylet is retracted, and a positron-emitting rod is inserted into the sheath. The positron-emitting rod can then be detected by the gamma camera to confirm proper placement of the sheath into the lesion. Assuming proper placement was achieved, a biopsy core can then be extracted through the sheath using a vacuum-assisted device, and a marker is placed through the sheath at the biopsy site. However, this approach has the disadvantages of requiring extra equipment such as the positron-emitting rod, and confirming proper placement of the needle with the positron-emitting rod only after the needle has already been positioned.

It would be desirable, therefore, to provide a way of performing biopsies and other medical procedures that use needles, and perform them during molecular imaging, which overcomes the disadvantages mentioned above.

### SUMMARY OF THE INVENTION

In one embodiment, a method is provided for needle guidance in molecular imaging, comprising the steps of: providing a molecular imaging system having one or more radiation detectors for detecting radiation emitted from tissue of a patient within a region of interest, a hollow needle for performing a medical procedure on the tissue, and a radioisotope in substantially liquid form; introducing the radioisotope into a lumen of the needle; and detecting radiation emitted from the radioisotope contained in the lumen of the needle using the molecular imaging system. The molecular imaging system may further include a display, and the region of interest may include one or both breasts. The medical procedure may be a biopsy, an injection of medication, and/or the implantation of markers or radiotherapy seeds. The method may further comprise one or more of the steps of injecting the radioisotope into the patient, detecting radiation emitted from the radioisotope contained in the tissue within the region of interest using the molecular imaging system, displaying a combined image of the radiation detected from the radioisotope contained in the needle and in the patient tissue, guiding the needle to perform the medical procedure on the tissue by using the combined image or the radiation detected from the needle, and determining whether the tip of the lumen is displayed on the display, and if not, then filling in the tip of the lumen on the display.

In another embodiment, a method is provided for needle guidance in molecular imaging, comprising the steps of: providing a molecular imaging system having one or more radiation detectors and a display for displaying a functional image of tissue of a patient within a region of interest, a hollow needle for performing a medical procedure on the tissue, and a radioisotope in substantially liquid form; injecting the radioisotope into the patient; introducing the radioisotope into the lumen of the needle; detecting radiation emitted from the radioisotope contained in the lumen of the needle and in the tissue within the region of interest using the molecular imaging system; and displaying a combined image of the radiation detected from the radioisotope contained in the needle and in the patient tissue. The region of interest may include one or both breasts, and the medical procedure may be a biopsy, an injection of medication, and/or the implantation of markers or radiotherapy seeds. The method may further comprise one or more of the steps of guiding the needle to perform the medical procedure on the tissue by using the combined image or the radiation detected from the needle, and determining whether the tip of the lumen is displayed on the display, and if not, then filling in the tip of the lumen on the display.

In yet another embodiment, a method is provided for needle guidance in molecular imaging, comprising the steps of: providing a molecular imaging system having one or more radiation detectors and a display for displaying a functional image of tissue of a patient within a region of interest, a hollow needle for performing a medical procedure on the tissue, and a radioisotope in substantially liquid form; injecting the radioisotope into the patient; introducing the radioisotope into the lumen of the needle; detecting radiation emitted from the radioisotope contained in the lumen of the needle and in the tissue within the region of interest using the molecular imaging system; displaying a combined image of the radiation detected from the radioisotope contained in the needle and in the patient tissue; and guiding the needle to perform the medical procedure on the tissue by using the combined image. The region of interest may include one or both breasts, and the medical procedure may be a biopsy, an injection of medication, and/or the implantation of markers or radiotherapy seeds. The method may further comprise one or more of the steps of guiding the needle to perform the medical procedure on the tissue by using the combined image or the radiation detected from the needle, and determining whether the tip of the lumen is displayed on the display, and if not, then filling in the tip of the lumen on the display.

### BRIEF DESCRIPTION OF THE DRAWINGS

FIG. 1 illustrates one approach of performing a biopsy according to the prior art.

FIG. 2 is a flowchart showing the steps of one embodiment of the present invention.

FIG. 3 is a flowchart showing the steps of another embodiment of the present invention.

FIG. 4 is a combined image of the tissue and needle according to various embodiments of the present invention.

FIG. 5 is a diagram showing relationships among aspects of manual and robotic needle movements according to various embodiments of the present invention.

FIG. 6 is a flowchart showing the steps of yet another embodiment of the present invention.

FIG. 7 is a portion of the combined image according to various embodiments of the present invention, showing (a) a non-detected/non-displayed tip of the needle and (b) a filled-in image of the needle.

FIG. 8 is a system according to various embodiments of the present invention in which the RP-filled needle is not yet within the detector field of view.

FIG. 9 is a system according to various embodiments of the present invention in which the RP-filled needle is within the detector field of view.

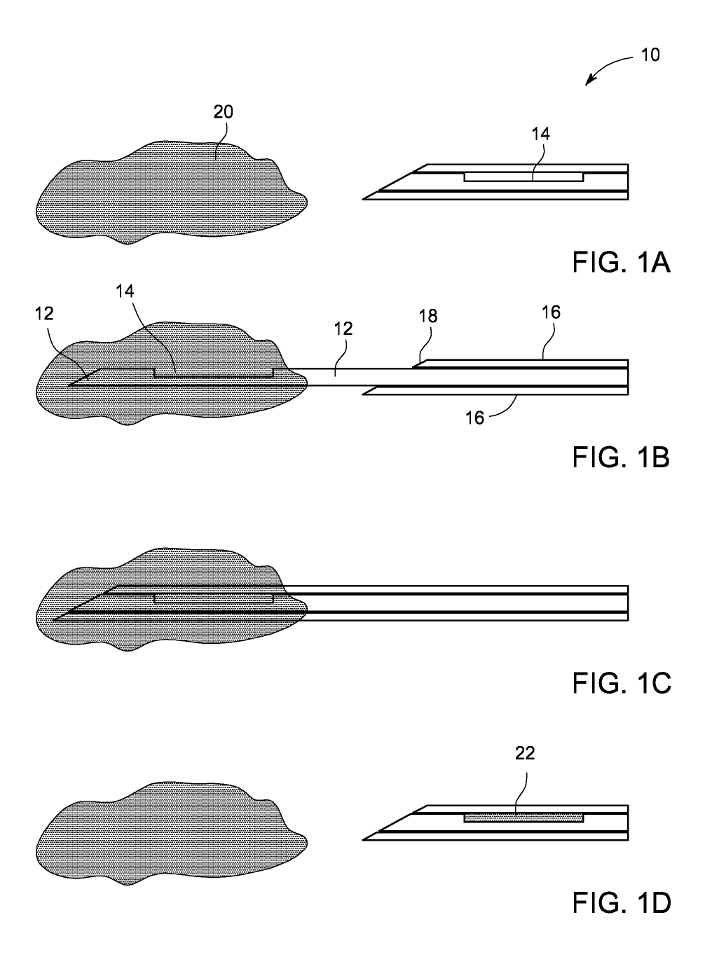

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware or circuitry. For example, one or more of the functional blocks (e.g., processors or memories) may be implemented in a single piece of hardware (e.g., a general purpose signal processor or random access memory, hard disk, or the like); likewise, a single functional block may be implemented in more than one piece of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, any references to a particular embodiment of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments of the invention provide a system and method for performing needle guidance into an anatomy of interest for molecular imaging. A technical effect of the various embodiments is to provide a molecular imaging system and method that is configured to allow imaging of a needle containing an RP in order to facilitate needle guidance for medical procedures, such as needle biopsies.

To assist the reader in understanding the embodiments of the present invention that are disclosed, all reference numbers used herein are summarized below, along with the elements they represent:
10 Needle
12 Inner body
14 Compartment
16 Outer sheath
18 Leading edge of sheath
20 Lesion
22 Lesion sample
30 Molecular imaging system
32 Radiation detector
33 Collimator
34 Radiation emitted from tissue
36 Tissue
38 Region of interest
40 Needle
42 Lumen
43 Reservoir/syringe barrel
44 Radioisotope/RP
46 Processing and control system
48 Display
50 Radiation emitted from needle
52 Combined image
54 Image of tissue (radiation emitted from tissue)
56 Image of needle (radiation emitted from needle)
58 Tip of needle
60 Projected trajectory of needle
62 Detected/displayed portion of needle
64 Non-detected/non-displayed portion of needle
66 Filled-in image of needle
68 Compression paddle
100-190 Steps associated with various embodiments of the present invention

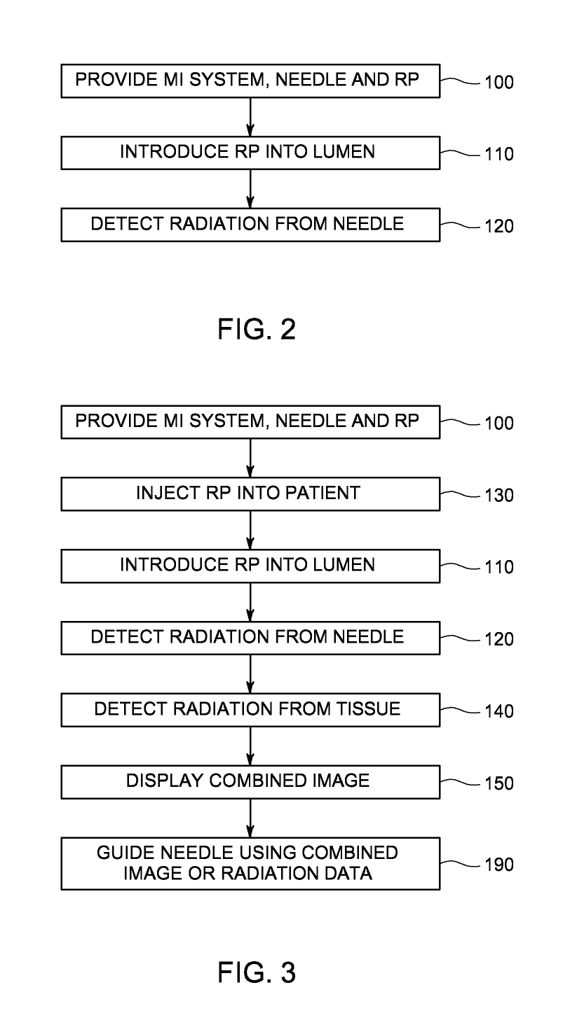

Referring now to the drawings, FIG. 2 shows a flowchart illustrating the steps of one embodiment of the present invention. In this embodiment, a method is provided for needle guidance in molecular imaging, comprising the steps of: (100) providing a molecular imaging system 30 having one or more radiation detectors 32 for detecting radiation 34 emitted from tissue 36 of a patient within a region of interest 38, a hollow needle 40 for performing a medical procedure on the tissue, and a radioisotope or RP 44 in substantially liquid form that is suitable for the type of molecular imaging desired; (110) introducing the radioisotope into a lumen 42 of the needle; and (120) detecting radiation 50 emitted from the radioisotope contained in the lumen of the needle using the molecular imaging system.

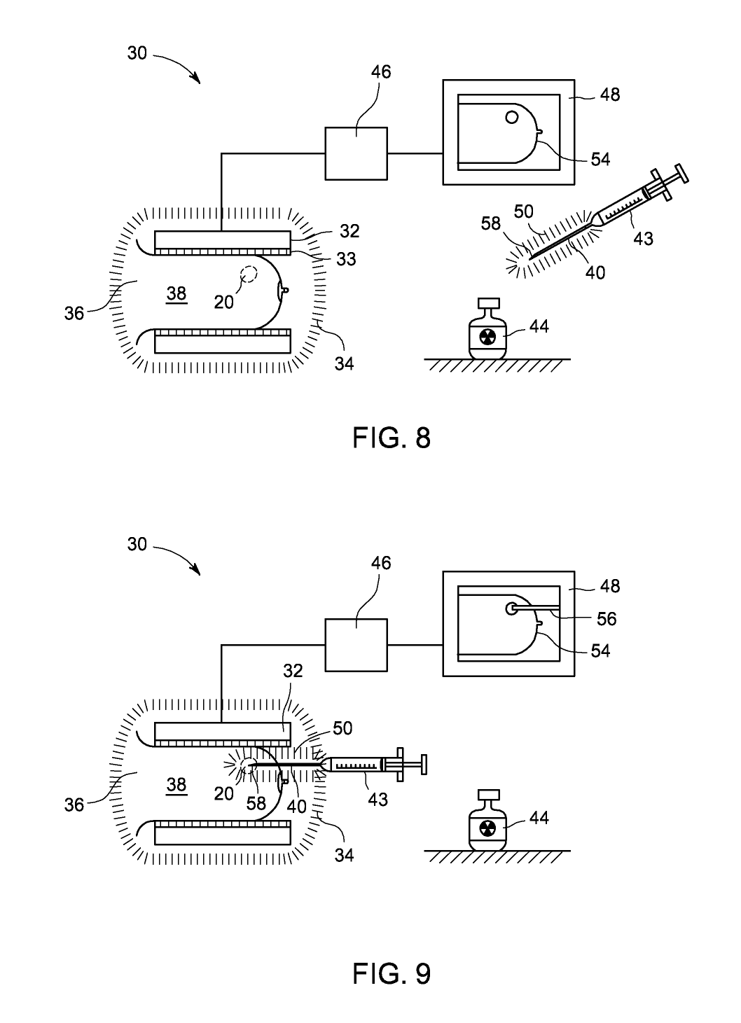

As shown in FIGS. 8 and 9, the radiation detector(s) 32 are gamma cameras (e.g., either a single one, or two arranged in H-mode, L-mode or L mode at oblique (non-90 degree angle) configurations, etc.) or other suitable detectors (e.g., PET detectors) for detecting gamma photons for molecular imaging, and may optionally include a collimator 33 for one or more of the detectors. The molecular imaging system 30 may be configured to perform nuclear medicine, SPECT, PET, PEM (positron emission mammography), scintimammography, or other types of molecular imaging scans. The system 30 may further include a display 48, and a processing and control system 46 for controlling the movement of the detector(s) 32 and/or the patient bed or seat (not shown) and for data and image processing. The region of interest 38 may include one or both breasts, such as in the case of molecular breast imaging, but may also comprise other areas of anatomy. The medical procedure may be a needle biopsy, an injection of medication or other delivery of therapy, and/or the implantation or extraction of markers or radiotherapy seeds.

The step (110) of introducing the radioisotope 44 into a lumen 42 of the needle 40 may involve using a specially produced and sealed or otherwise normal biopsy syringe/needle combination and drawing the liquid radioisotope up through the needle from a container by retracting the plunger, or by having the radioisotope in the reservoir/barrel 43 and advancing the plunger so as to force the radioisotope toward the tip 58, or by opening and closing the sheath 16 while the needle tip is submerged in a reservoir of liquid radioisotope if there is no plunger, thereby capturing radioisotope in the closed compartment 14. The step (120) of detecting radiation 50 emitted from the radioisotope contained in the lumen/needle involves placing the needle in the field of view of the detectors 32 so that the molecular imaging system can detect the radiation being emitted from the radioisotope in the needle, thereby enabling the needle to be imaged.

As shown in FIG. 3, the embodiment may further comprise one or more of the steps of (130) injecting the radioisotope 44 into the patient, (140) detecting radiation 34 emitted from the radioisotope contained in the tissue 36 within the region of interest using the molecular imaging system, (150) displaying a combined image 52 of the radiation 34/50 detected from the radioisotope contained in the needle 40/42 and in the patient tissue 36, and (190) guiding the needle 40 to perform the medical procedure on the tissue by using the combined image 52 on the display 48 or by using the radiation 34 detected from the needle. The step (130) of injecting the radioisotope into the patient may comprise intravenous injection of an appropriate dose suitable for the desired imaging procedure. Once the dose has been administered, it is likely that the needle used for the injection will still be filled with radioisotope, so this same needle may be used for the fine needle biopsy or other medical procedure. Thus, once the step (130) of injecting the RP into the patient is completed, if the same needle is subsequently used for the biopsy or other medical procedure, then it is likely that the step (110) of introducing RP into the lumen is simultaneously accomplished. However, it is also possible that two different needles are used—one for injecting RP into the patient, and another for the biopsy or other medical procedure—but there is potential workflow synergy and reduced expense by using a single needle for both injecting the RP into the patient and performing the medical procedure on the patient. In either case, one advantage of this embodiment of the present invention is that regular needles may be used, instead of needles having radioactive metals or coatings which are much more expensive and difficult to dispose of.

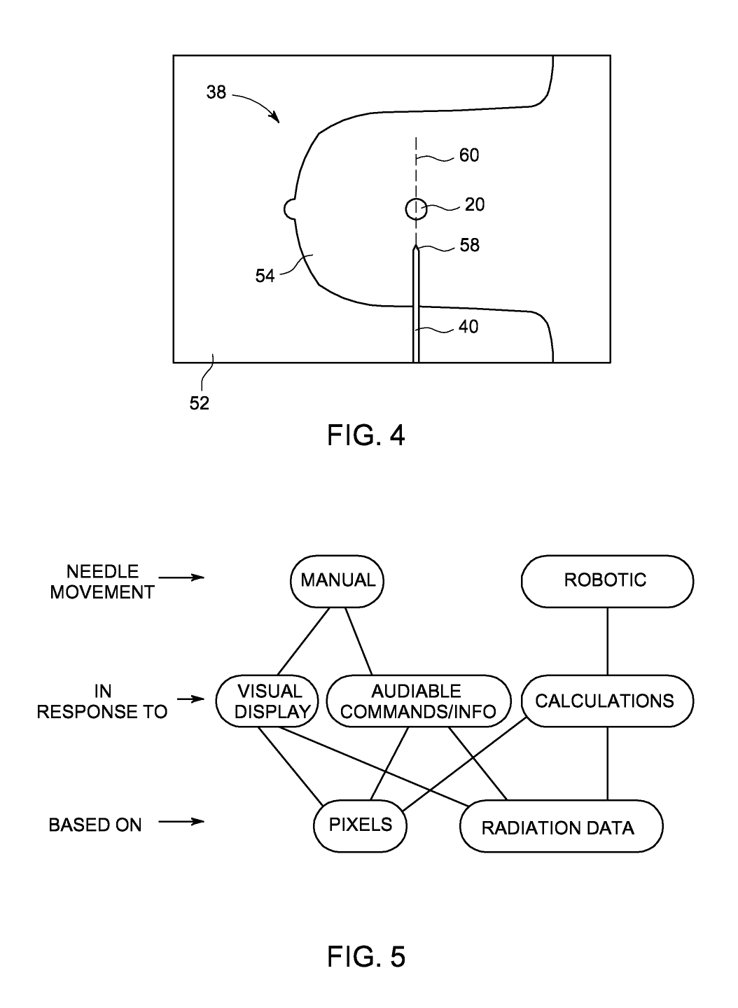

The step (140) of detecting radiation emitted from the tissue may be performed at the same time as the step (120) of detecting radiation from the radioisotope-filled needle, by using the one or more detectors 32 of the molecular imaging system. The radiation 34 detected from the needle 40/42 and the radiation 50 detected from the tissue 36 may be processed by the processing and control system 46 and displayed as a combined image 52 showing both the region of interest 38 and the needle 40/42 in one view, as illustrated in FIG. 4. Here, a cranial-caudal view of a compressed breast is shown. An image 54 of the tissue (i.e., the radiation 50 detected from the tissue) is shown, as well as an image 56 of the needle (i.e., of the radiation 34 detected from the lumen of the needle), together providing a combined image 52. Although shown here as only one screen, the combined image 52 may also be displayed as two or more screens or images, such as when L-mode nuclear imaging is used which can provide not only information in one plane (e.g., in the x- and y-directions) but also depth information (e.g., in the z-direction).

The step (190) of guiding the needle to perform the biopsy or other medical procedure can be done by various approaches, as illustrated in FIG. 5. The needle may be guided into the tissue/lesion either manually (by hand by a human operator) or robotically, and these movements may be based on the pixels displayed in the combined image or based on data from the radiation 34/50 detected. (Note that the pixels displayed in the combined image are themselves the result of calculations using data based on the detected radiation 34/50.) If the needle guidance is done manually, the operator can watch the combined image 52 on the display 48 as a guide for directing the needle into the tissue/lesion. The operator can also guide the needle based on commands and information provided by the processing and control system 46 on the display (e.g., color coding, projected needle trajectory, numerical readouts of needle tip depth/position, text prompts, flashing icons/text, etc.) and/or audibly (e.g., voice commands, sound effects, etc.) The trajectory 60 of the needle (i.e., the direction the needle is pointing in) can be projected onto the display to assist the operator in pointing and guiding the needle manually. (This projection 60 can be calculated in software by the processing and control system 46 based on the data from the detected needle radiation.) For manual movement, the visual and audible prompts, images and information can be based on the pixels/pixel data, the detected radiation/radiation data, or both. If the needle guidance is done robotically, the movements of the needle can be in response to calculations performed in the processing and control system 46 based on the pixel data, the radiation data or both. These calculations can be of various positions (of the lesion, needle, skin surface, etc.), trajectories (of the needle, needle movement mechanisms, etc.), and the like for controlled robotic movement of the needle. In both the manual or robotic approaches, it may also be useful to utilize the pixel data from the image 54 of the tissue and/or the data from the detected tissue radiation 34 as a frame of reference for the needle movements and/or for the visual and audible prompts and information. Additionally, it may be useful to utilize one or more external frames of reference (e.g., scanner coordinates, stereotactic coordinates, wired or wirelessly transmitted position coordinates from infrared, electromagnetic or other sensors, etc.) for the same purpose.

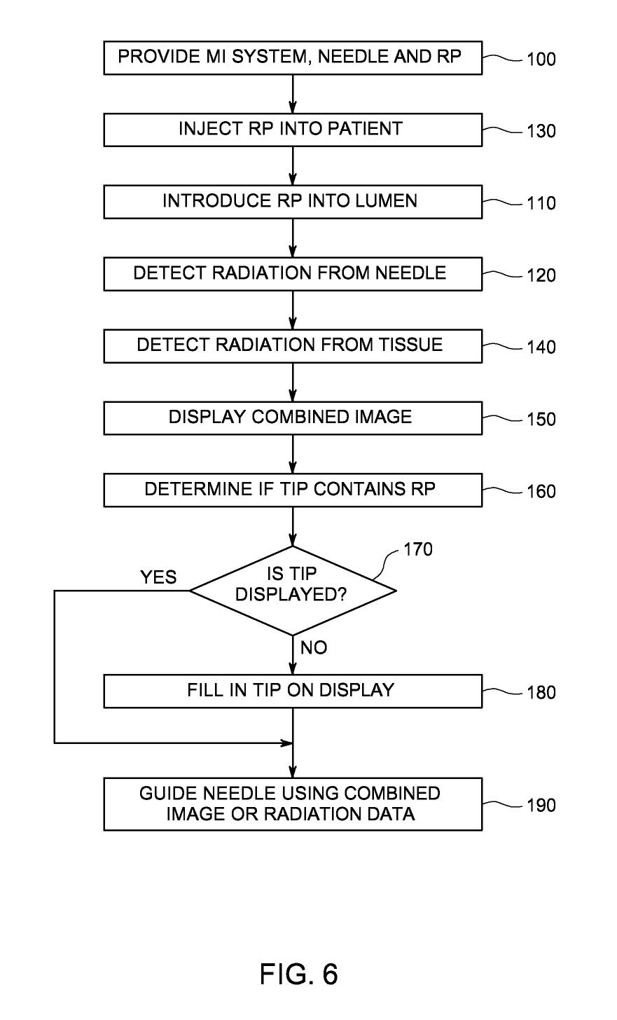

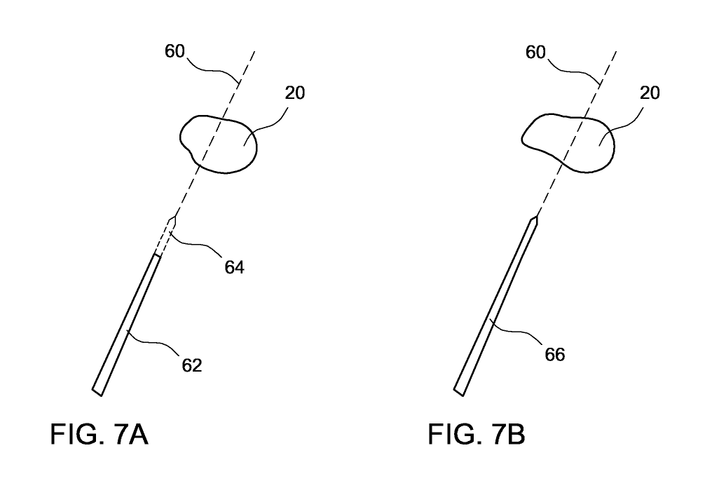

FIGS. 6 and 7 illustrate the optional additional steps (160/170) of determining whether the tip 58 of the lumen contains radioisotope (i.e., does the tip appear on the display 48), and if not, then (180) filling in the tip of the lumen on the display 48/52. Step (160) may be done by various approaches, such as advancing the plunger on the syringe containing the radioisotope and the operator visually confirming that radioisotope drips from the needle tip (thereby confirming that radioisotope extends through the lumen to the tip), or by placing the needle in the detector field of view and viewing the image 56 of the needle on the display to confirm that at least the tip appears. (The needle/syringe can be loaded into a simple fixture having a radioactive marker at or adjacent to the tip of the needle, and the loaded fixture placed into the detector field of view so that the operator can confirm on the display that the image of the needle includes at least the tip.) Since the tip 58 of the needle is where the extraction of tissue, delivery of medicine, etc. occurs, it may be desired that at least the tip is visible on the display. If it is confirmed that the tip is not filled with radioisotope (e.g., the tip is not detected and displayed) such as is illustrated in FIG. 7(a), then (180) the non-detected/non-displayed tip 64 may be filled in 66 on the display using software (e.g., based on known geometry/measurements of the syringe and needle as compared against the abovementioned fixture or against other known landmarks or features in the tissue image 54), as illustrated in FIG. 7(b). Of course, any other non-detected/non-displayed portions 64 besides the tip may also be filled in using the same approach. It may also be desirable that at least the tip and some of the needle immediately adjacent to the tip are visible on the display, so that the detected or imaged needle can be used to project a line segment 60 outward from the tip on the display so that the trajectory 60 or direction that the needle is pointing in can be seen by the operator on the display.

FIGS. 8 and 9 show a system according to various embodiments of the present invention. In FIG. 8, the RP-filled needle is not yet within the detector field of view, while in FIG. 9 the RP-filled needle is within the detector field of view. Although reference is made above to FIGS. 8 and 9 in connection with a first embodiment of the present invention (FIG. 2), the system shown in FIGS. 8 and 9 may also apply to other embodiments and aspects of the present invention (e.g., also FIGS. 3-7). In FIG. 8, a syringe with needle 40 is shown having RP 44 filling the lumen of the needle, with radiation 50 in the form of gamma photons being emitted from the needle. It should be noted that although a syringe with barrel 43 is shown for the purpose of illustration in FIGS. 8 and 9, other suitable alternatives may also be provided for holding the RP in fluid communication with the lumen of the needle, such as tubing, rigid barrels attached to pumps, biopsy devices, etc. In FIG. 9, the needle 40 (and its attached barrel, tubing, etc.) is placed into the detector field of view, and thus the needle becomes visible on the display 48 due to the radiation 50 from the needle that is now detected.

In each of the embodiments described, the radioisotope/RP should be one having a relatively short half-life so that the radiation level of the needle can safely diminish to facilitate handling and disposal. For example, technecium-99m, having a half-life of about six hours, may be used. Additionally, the steps for executing the methods described herein may be embodied in computer readable media, software and the like.

The above description is intended to be illustrative, and not restrictive. While the invention has been described in terms of various specific embodiments, those skilled in the art will recognize that the invention can be practiced with modification within the spirit and scope of the claims. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to illustrate the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable those skilled in the art to practice the invention, including making and using any devices or systems thereof and performing any methods thereof. It is the following claims, including all equivalents, which define the scope of the present invention.

1. A method for needle guidance in molecular imaging, comprising the steps of:
providing a molecular imaging system having one or more radiation detectors for detecting radiation emitted from tissue of a patient within a region of interest, a hollow needle for performing a medical procedure on the tissue, and a radioisotope in substantially liquid form;
introducing the radioisotope into a lumen of the needle; and
detecting radiation emitted from the radioisotope contained in the lumen of the needle using the molecular imaging system.

2. A method according to claim 1, further comprising the step of injecting the radioisotope into the patient.

3. A method according to claim 2, further comprising the step of detecting radiation emitted from the radioisotope contained in the tissue within the region of interest using the molecular imaging system.

4. A method according to claim 3, wherein the molecular imaging system includes a display, and further comprising the step of displaying a combined image of the radiation detected from the radioisotope contained in the needle and in the patient tissue.

5. A method according to claim 4, further comprising the step of guiding the needle to perform the medical procedure on the tissue by using the combined image or the radiation detected from the radioisotope contained in the needle.

6. A method according to claim 1, wherein the medical procedure is a biopsy.

7. A method according to claim 1, wherein the medical procedure is an injection of medication or the implantation or extraction of markers or radiotherapy seeds

8. A method according to claim 1, wherein the region of interest is one or both breasts.

9. A method according to claim 1, further comprising the steps of determining whether the tip of the lumen is displayed on the display, and if not, then filling in the tip of the lumen on the display.

10. A method for needle guidance in molecular imaging, comprising the steps of:
providing a molecular imaging system having one or more radiation detectors and a display for displaying a functional image of tissue of a patient within a region of interest, a hollow needle for performing a medical procedure on the tissue, and a radioisotope in substantially liquid form;
injecting the radioisotope into the patient;
introducing the radioisotope into the lumen of the needle;
detecting radiation emitted from the radioisotope contained in the lumen of the needle and in the tissue within the region of interest using the molecular imaging system; and
displaying a combined image of the radiation detected from the radioisotope contained in the needle and in the patient tissue.

11. A method according to claim 10, further comprising the step of guiding the needle to perform the medical procedure on the tissue by using the combined image or the radiation detected from the radioisotope contained in the needle.

12. A method according to claim 11, wherein the medical procedure is a biopsy.

13. A method according to claim 10, wherein the medical procedure is an injection of medication or the implantation or extraction of markers or radiotherapy seeds

14. A method according to claim 10, wherein the region of interest is one or both breasts.

15. A method according to claim 10, further comprising the steps of determining whether the tip of the lumen is displayed on the display, and if not, then filling in the tip of the lumen on the display.

16. A method for needle guidance in molecular imaging, comprising the steps of:
providing a molecular imaging system having one or more radiation detectors and a display for displaying a functional image of tissue of a patient within a region of interest, a hollow needle for performing a medical procedure on the tissue, and a radioisotope in substantially liquid form;
injecting the radioisotope into the patient;
introducing the radioisotope into the lumen of the needle;
detecting radiation emitted from the radioisotope contained in the lumen of the needle and in the tissue within the region of interest using the molecular imaging system;
displaying a combined image of the radiation detected from the radioisotope contained in the needle and in the patient tissue; and
guiding the needle to perform the medical procedure on the tissue by using the combined image or the radiation detected from the radioisotope contained in the needle.

17. A method according to claim 16, wherein the medical procedure is a biopsy.

18. A method according to claim 16, wherein the medical procedure is an injection of medication or the implantation or extraction of markers or radiotherapy seeds

19. A method according to claim 16, wherein the region of interest is one or both breasts.

20. A method according to claim 16, further comprising the steps of determining whether the tip of the lumen is displayed on the display, and if not, then filling in the tip of the lumen on the display.
